(19) United States
(12) Patent Application Publication
(10) Pub. No.: US 2023/0223124 A1
(43) Pub. Date: Jul. 13, 2023

(54) INFORMATION PROCESSING APPARATUS, INFORMATION PROCESSING METHOD, AND INFORMATION PROCESSING PROGRAM

(71) Applicant: FUJIFILM Corporation, Tokyo (JP)

(72) Inventor: Keigo NAKAMURA, Tokyo (JP)

(73) Assignee: FUJIFILM Corporation, Tokyo (JP)

(21) Appl. No.: 18/149,159

(22) Filed: Jan. 3, 2023

(30) Foreign Application Priority Data
Jan. 11, 2022 (JP) 2022-002561

(51) Int. Cl. G16H 15/00; G16H 30/40

(52) U.S. Cl. CPC G16H 15/00; G16H 30/40

(57) ABSTRACT

An information processing apparatus comprising at least one processor, wherein the at least one processor is configured to: acquire a plurality of pieces of element information related to a medical image; divide the plurality of pieces of element information into groups; and generate summary information in which a summary of the element information included in the group is associated with the group.

### CROSS-REFERENCE TO RELATED APPLICATIONS

This application claims priority from Japanese Application No. 2022-002561, filed on Jan. 11, 2022, the entire disclosure of which is incorporated herein by reference.

### BACKGROUND

### Technical Field

The present disclosure relates to an information processing apparatus, an information processing method, and an information processing program.

### Related Art

In the related art, image diagnosis is performed using medical images obtained by imaging apparatuses such as computed tomography (CT) apparatuses and magnetic resonance imaging (MRI) apparatuses. In addition, medical images are analyzed via computer aided detection/diagnosis (CAD) using a discriminator in which learning is performed by deep learning or the like, and regions of interest including structures, lesions, and the like included in the medical images are detected and/or diagnosed. The medical images and analysis results via CAD are transmitted to a terminal of a healthcare professional such as a radiologist who interprets the medical images. The healthcare professional such as a radiologist interprets the medical image by referring to the medical image and analysis result using his or her own terminal and creates an interpretation report.

In addition, various methods have been proposed to support the creation of interpretation reports in order to reduce the burden of the interpretation work of a radiologist. For example, JP2019-153250A discloses a technique for creating an interpretation report based on a keyword input by a radiologist and an analysis result of a medical image. In the technique described in JP2019-153250A, a sentence to be included in the interpretation report is created by using a recurrent neural network trained to generate a sentence from input characters.

In addition, for example, JP2015-179319A discloses a technique for improving the viewability of an interpretation report by displaying information indicating each of a diagnosis disease name, a lesion associated with the diagnosis disease name, and a report related to the lesion in association with each other.

In recent years, the amount of information of the analysis result obtained from the medical image has been increasing with the increase in the performance of the imaging apparatus and the performance of the CAD. In a case where a large amount of analysis results are obtained from the medical image, presenting all of the analysis results makes the confirmation work complicated for the creator of the interpretation report. Therefore, there is a demand for a technique that allows an overall overview of analysis results obtained from medical images to be easily viewed.

### SUMMARY

The present disclosure provides an information processing apparatus, an information processing method, and an information processing program capable of supporting creation of interpretation reports.

According to a first aspect of the present disclosure, there is provided an information processing apparatus comprising at least one processor, in which the processor is configured to acquire a plurality of pieces of element information related to a medical image, divide the plurality of pieces of element information into groups, and generate summary information in which a summary of the element information included in the group is associated with the group.

In the first aspect, the processor may be configured to acquire the element information related to each of a plurality of regions of interest included in the medical image, and divide the plurality of pieces of element information into groups for each of the regions of interest.

In the first aspect, the processor may be configured to collect the element information related to each of two or more similar regions of interest among the plurality of regions of interest in the same group.

In the first aspect, the processor may be configured to generate the summary information based on the element information common to each of the regions of interest collected in the group.

In the first aspect, the processor may be configured to generate the summary information based on the element information indicating at least one of a size or a position of the region of interest, which is common to each of the regions of interest collected in the group.

In the first aspect, the processor may be configured to generate the summary information based on the element information having a highest degree of malignancy among the element information related to each of the regions of interest collected in the group.

In the first aspect, the processor may be configured to generate the summary information based on the number of regions of interest collected in the group.

In the first aspect, the processor may be configured to acquire the element information related to each of a plurality of the medical images.

In the first aspect, the processor may be configured to divide the plurality of pieces of element information into groups for each of the medical images.

In the first aspect, the processor may be configured to collect the element information related to each of two or more medical images having at least one of the same imaging method or imaging condition in the same group.

In the first aspect, the plurality of medical images may include images captured at different imaging points in time, and the processor may be configured to collect the element information related to each of the plurality of medical images having the same region of interest of the same subject as an imaging target and captured at different imaging points in time in the same group.

In the first aspect, the processor may be configured to display the summary information for each group on a display in a tabular format.

In the first aspect, the processor may be configured to assign the summary information to regions corresponding to each group on a schema and display the summary information on a display.

In the first aspect, the processor may be configured to set at least one of the medical images related to the element information included in the group as a representative image, and display the summary information for each group and the representative image on a display in association with each other.

In the first aspect, status information indicating a status of a work performed with regard to the element information collected in the group may be assigned to each group, and the processor may be configured to display the summary information for each group and the status information assigned to the group on a display in association with each other.

In the first aspect, the processor may be configured to acquire the medical image, and generate the element information based on the acquired medical image.

In the first aspect, the processor may be configured to detect a region of interest from the acquired medical image, and generate the element information related to the detected region of interest.

In the first aspect, the element information may be information indicating at least one of a name, a property, a measured value, a position, or an estimated disease name related to a region of interest included in the medical image, or an imaging method, an imaging condition, or an imaging date and time related to imaging of the medical image, and the region of interest may be at least one of a region of a structure included in the medical image or a region of an abnormal shadow included in the medical image.

According to a second aspect of the present disclosure, there is provided an information processing method comprising acquiring a plurality of pieces of element information related to a medical image, dividing the plurality of pieces of element information into groups, and generating summary information in which a summary of the element information included in the group is associated with the group.

According to a third aspect of the present disclosure, there is provided an information processing program causing a computer to execute a process comprising acquiring a plurality of pieces of element information related to a medical image, dividing the plurality of pieces of element information into groups, and generating summary information in which a summary of the element information included in the group is associated with the group.

According to the above aspects, the information processing apparatus, information processing method, and information processing program of the present disclosure can support the creation of interpretation reports.

### BRIEF DESCRIPTION OF THE DRAWINGS

FIG. 1 is a diagram showing an example of a schematic configuration of an information processing system.

FIG. 2 is a diagram showing an example of a medical image.

FIG. 3 is a diagram showing an example of a medical image.

FIG. 4 is a block diagram showing an example of a hardware configuration of an information processing apparatus.

FIG. 5 is a block diagram showing an example of a functional configuration of the information processing apparatus.

FIG. 6 is a diagram showing an example of a detector.

FIG. 7 is a diagram showing an example of element information.

FIG. 8 is a diagram showing an example of grouping.

FIG. 9 is a diagram showing an example of summary information.

FIG. 10 is a diagram showing an example of a screen displayed on a display.

FIG. 11 is a diagram showing an example of a screen displayed on a display.

FIG. 12 is a flowchart showing an example of information processing.

FIG. 13 is a diagram showing an example of a screen according to Modification Example 1.

FIG. 14 is a diagram showing an example of a screen according to Modification Example 2-1.

FIG. 15 is a diagram showing an example of a screen according to Modification Example 2-2.

FIG. 16 is a diagram showing an example of a screen according to Modification Example 2-3.

FIG. 17 is a diagram showing an example of a screen according to Modification Example 2-4.

FIG. 18 is a diagram showing an example of a screen according to Modification Example 2-4.

FIG. 19 is a diagram showing an example of a screen according to Modification Example 3.

FIG. 20 is a diagram showing an example of a screen according to Modification Example 4-1.

FIG. 21 is a diagram showing an example of a screen according to Modification Example 4-2.

FIG. 22 is a diagram showing an example of a screen according to Modification Example 5.

FIG. 23 is a diagram showing an example of a screen according to Modification Example 5.

FIG. 24 is a diagram showing an example of a screen according to Modification Example 6-1.

FIG. 25 is a diagram showing an example of a screen according to Modification Example 6-1.

FIG. 26 is a diagram showing an example of a screen according to Modification Example 6-3.

FIG. 27 is a diagram showing an example of a screen according to Modification Example 6-3.

### DETAILED DESCRIPTION

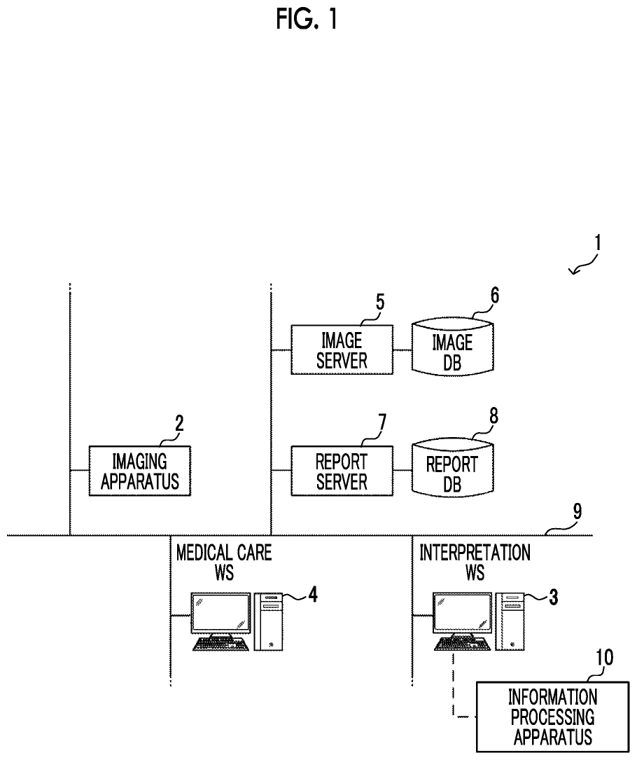

Hereinafter, an embodiment of the present disclosure will be described with reference to the drawings. First, a configuration of an information processing system 1 to which an information processing apparatus of the present disclosure is applied will be described. FIG. 1 is a diagram showing a schematic configuration of the information processing system 1. The information processing system 1 shown in FIG. 1 performs imaging of an examination target part of a subject and storing of a medical image acquired by the imaging based on an examination order from a doctor in a medical department using a known ordering system. In addition, the information processing system 1 performs an interpretation work of a medical image and creation of an interpretation report by a radiologist and viewing of the interpretation report by a doctor of a medical department that is a request source.

As shown in FIG. 1, the information processing system 1 includes an imaging apparatus 2, an interpretation work station (WS) 3 that is an interpretation terminal, a medical care WS 4, an image server 5, an image database (DB) 6, a report server 7, and a report DB 8. The imaging apparatus 2, the interpretation WS 3, the medical care WS 4, the image server 5, the image DB 6, the report server 7, and the report DB 8 are connected to each other via a wired or wireless network 9 in a communicable state.

Each apparatus is a computer on which an application program for causing each apparatus to function as a component of the information processing system 1 is installed. The application program may be recorded on, for example, a recording medium, such as a digital versatile disc (DVD) or a compact disc read only memory (CD-ROM), and distributed, and be installed on the computer from the recording medium. In addition, the application program may be stored in, for example, a storage apparatus of a server computer connected to the network 9 or in a network storage in a state in which it can be accessed from the outside, and be downloaded and installed on the computer in response to a request.

The imaging apparatus 2 is an apparatus (modality) that generates a medical image T showing a diagnosis target part of the subject by imaging the diagnosis target part. Specifically, examples of the imaging apparatus 2 include a simple X-ray imaging apparatus, a CT apparatus, an MRI apparatus, a positron emission tomography (PET) apparatus, and the like. The medical image generated by the imaging apparatus 2 is transmitted to the image server 5 and is saved in the image DB 6.

The interpretation WS 3 is a computer used by, for example, a healthcare professional such as a radiologist of a radiology department to interpret a medical image and to create an interpretation report, and encompasses an information processing apparatus 10 according to the present embodiment. In the interpretation WS 3, a viewing request for a medical image to the image server 5, various image processing for the medical image received from the image server 5, display of the medical image, and input reception of a sentence regarding the medical image are performed. In the interpretation WS 3, an analysis process for medical images, support for creating an interpretation report based on the analysis result, a registration request and a viewing request for the interpretation report to the report server 7, and display of the interpretation report received from the report server 7 are performed. The above processes are performed by the interpretation WS 3 executing software programs for respective processes.

The medical care WS 4 is a computer used by, for example, a healthcare professional such as a doctor in a medical department to observe a medical image in detail, view an interpretation report, create an electronic medical record, and the like, and is configured to include a processing apparatus, a display apparatus such as a display, and an input apparatus such as a keyboard and a mouse. In the medical care WS 4, a viewing request for the medical image to the image server 5, display of the medical image received from the image server 5, a viewing request for the interpretation report to the report server 7, and display of the interpretation report received from the report server 7 are performed. The above processes are performed by the medical care WS 4 executing software programs for respective processes.

The image server 5 is a general-purpose computer on which a software program that provides a function of a database management system (DBMS) is installed. The image server 5 is connected to the image DB 6. The connection form between the image server 5 and the image DB 6 is not particularly limited, and may be a form connected by a data bus, or a form connected to each other via a network such as a network attached storage (NAS) and a storage area network (SAN).

The image DB 6 is realized by, for example, a storage medium such as a hard disk drive (HDD), a solid state drive (SSD), and a flash memory. In the image DB 6, the medical image acquired by the imaging apparatus 2 and accessory information attached to the medical image are registered in association with each other.

The accessory information may include, for example, identification information such as an image identification (ID) for identifying a medical image, a tomographic ID assigned to each tomographic image included in the medical image, a subject ID for identifying a subject, and an examination ID for identifying an examination. In addition, the accessory information may include, for example, information related to imaging such as an imaging method, an imaging condition, and an imaging date and time related to imaging of a medical image. The “imaging method” and “imaging condition” are, for example, a type of the imaging apparatus 2, an imaging part, an imaging protocol, an imaging sequence, an imaging method, the presence or absence of use of a contrast medium, a slice thickness in tomographic imaging, and the like. In addition, the accessory information may include information related to the subject such as the name, age, and gender of the subject.

In a case where the image server 5 receives a request to register a medical image from the imaging apparatus 2, the image server 5 prepares the medical image in a format for a database and registers the medical image in the image DB 6. In addition, in a case where the viewing request from the interpretation WS 3 and the medical care WS 4 is received, the image server 5 searches for a medical image registered in the image DB 6 and transmits the searched for medical image to the interpretation WS 3 and to the medical care WS 4 that are viewing request sources.

The report server 7 is a general-purpose computer on which a software program that provides a function of a database management system is installed. The report server 7 is connected to the report DB 8. The connection form between the report server 7 and the report DB 8 is not particularly limited, and may be a form connected by a data bus or a form connected via a network such as a NAS and a SAN.

The report DB 8 is realized by, for example, a storage medium such as an HDD, an SSD, and a flash memory. In the report DB 8, an interpretation report created in the interpretation WS 3 is registered.

Further, in a case where the report server 7 receives a request to register the interpretation report from the interpretation WS 3, the report server 7 prepares the interpretation report in a format for a database and registers the interpretation report in the report DB 8. Further, in a case where the report server 7 receives the viewing request for the interpretation report from the interpretation WS 3 and the medical care WS 4, the report server 7 searches for the interpretation report registered in the report DB 8, and transmits the searched for interpretation report to the interpretation WS 3 and to the medical care WS 4 that are viewing request sources.

The network 9 is, for example, a network such as a local area network (LAN) and a wide area network (WAN). The imaging apparatus 2, the interpretation WS 3, the medical care WS 4, the image server 5, the image DB 6, the report server 7, and the report DB 8 included in the information processing system 1 may be disposed in the same medical institution, or may be disposed in different medical institutions or the like. Further, the number of each apparatus of the imaging apparatus 2, the interpretation WS 3, the medical care WS 4, the image server 5, the image DB 6, the report server 7, and the report DB 8 is not limited to the number shown in FIG. 1, and each apparatus may be composed of a plurality of apparatuses having the same functions.

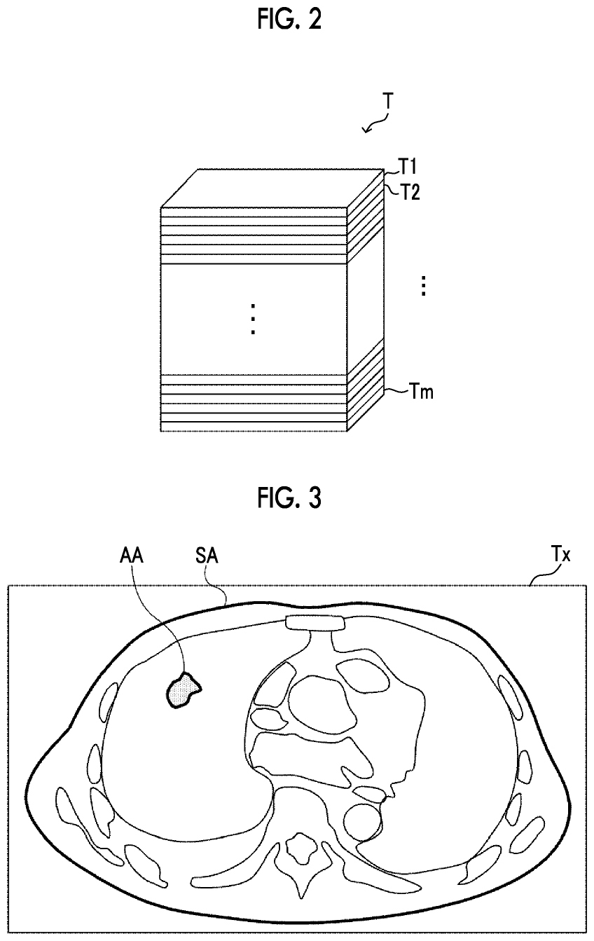

FIG. 2 is a diagram schematically showing an example of a medical image acquired by the imaging apparatus 2. The medical image T shown in FIG. 2 is, for example, a CT image consisting of a plurality of tomographic images T1 to Tm (m is 2 or more) representing tomographic planes from the chest to the lumbar region of one subject (human body). The plurality of tomographic images T1 to Tm are examples of a plurality of medical images of the present disclosure.

FIG. 3 is a diagram schematically showing an example of one tomographic image Tx out of the plurality of tomographic images T1 to Tm. The tomographic image Tx shown in FIG. 3 represents a tomographic plane including a lung. Each of the tomographic images T1 to Tm may include a region SA of a structure showing various organs of the human body (for example, lungs, livers, and the like), various tissues constituting various organs (for example, blood vessels, nerves, muscles, and the like), and the like. In addition, each tomographic image may include lesions (for example, nodules, tumors, injuries, defects, inflammation, and the like), and a region AA of an abnormal shadow showing regions obscured by imaging. In the tomographic image Tx shown in FIG. 3, the lung region is the region SA of the structure, and the nodule region is the region AA of the abnormal shadow. Hereinafter, at least one of the region SA of the structure or the region AA of the abnormal shadow is referred to as a “region of interest”. Note that one tomographic image may include a plurality of regions of interest.

Next, the information processing apparatus 10 according to the present embodiment will be described. The information processing apparatus 10 has a function of supporting the creation of an interpretation report based on a medical image. As described above, the information processing apparatus 10 is encompassed in the interpretation WS 3.

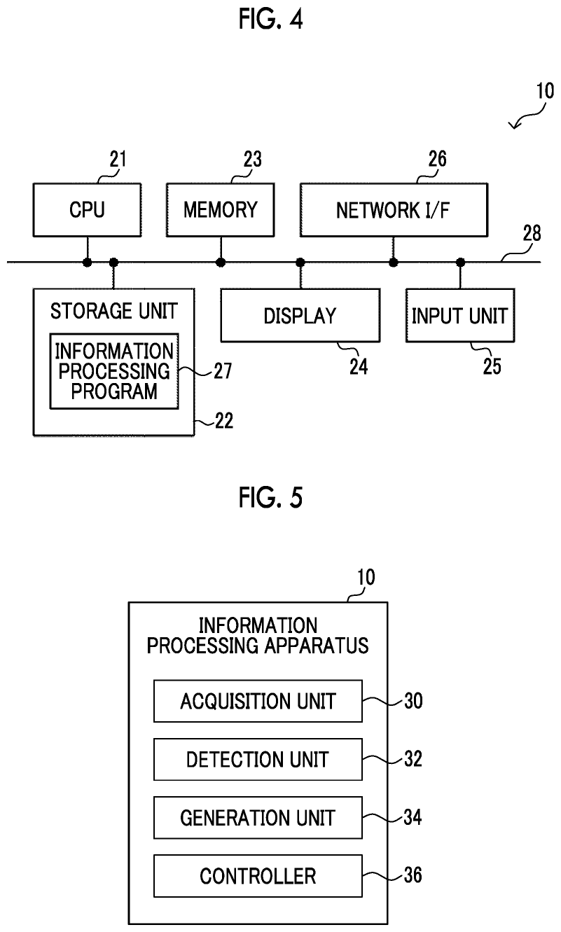

First, with reference to FIG. 4, an example of a hardware configuration of the information processing apparatus 10 according to the present embodiment will be described. As shown in FIG. 4, the information processing apparatus 10 includes a central processing unit (CPU) 21, a non-volatile storage unit 22, and a memory 23 as a temporary storage area. Further, the information processing apparatus 10 includes a display 24 such as a liquid crystal display, an input unit 25 such as a keyboard and a mouse, and a network interface (I/F) 26. The network I/F 26 is connected to the network 9 and performs wired or wireless communication. The CPU 21, the storage unit 22, the memory 23, the display 24, the input unit 25, and the network I/F 26 are connected to each other via a bus 28 such as a system bus and a control bus so that various types of information can be exchanged.

The storage unit 22 is realized by, for example, a storage medium such as an HDD, an SSD, and a flash memory. An information processing program 27 in the information processing apparatus 10 is stored in the storage unit 22. The CPU 21 reads out the information processing program 27 from the storage unit 22, loads the read-out program into the memory 23, and executes the loaded information processing program 27. The CPU 21 is an example of a processor of the present disclosure. As the information processing apparatus 10, for example, a personal computer, a server computer, a smartphone, a tablet terminal, a wearable terminal, or the like can be appropriately applied.

Next, with reference to FIG. 5, an example of a functional configuration of the information processing apparatus 10 according to the present embodiment will be described. As shown in FIG. 5, the information processing apparatus 10 includes an acquisition unit 30, a detection unit 32, a generation unit 34, and a controller 36. In a case where the CPU 21 executes the information processing program 27, the CPU 21 functions as the acquisition unit 30, the detection unit 32, the generation unit 34, and the controller 36.

The acquisition unit 30 acquires, from the image server 5, at least one medical image for which an interpretation report is to be created. In addition, the acquisition unit 30 may acquire a plurality of medical images related to the same subject. For example, a CT image consisting of a plurality of tomographic images as shown in FIG. 2 and FIG. 3 may be acquired. Further, for example, a plurality of medical images different in at least one of the type of imaging apparatuses 2, imaging conditions, or imaging methods, such as a simple CT image and a combination of a contrast CT image and an MRI image, may be acquired.

The detection unit 32 detects a region of interest included in the medical image acquired by the acquisition unit 30. In addition, the detection unit 32 may detect a plurality of regions of interest included in one medical image. In addition, in a case where a plurality of medical images are acquired by the acquisition unit 30, the detection unit 32 may detect a region of interest included in each of the plurality of medical images.

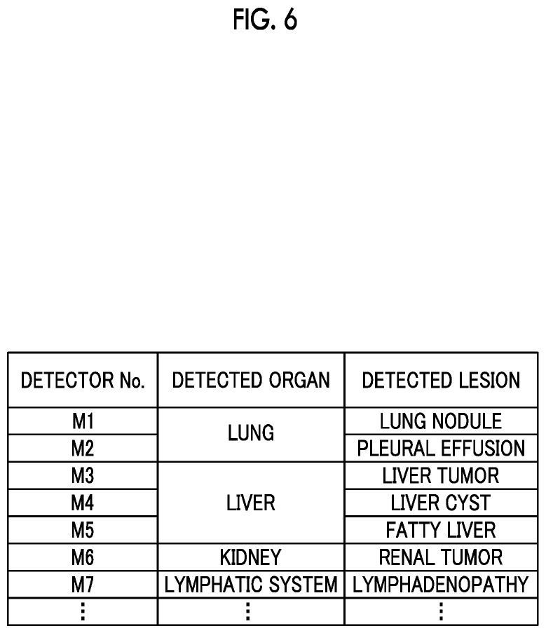

A specific example of a method of detecting a region of interest by the detection unit 32 will be described with reference to FIG. 6. FIG. 6 is a list of a plurality of types of detectors M1 to M7 for detecting various regions of interest from the medical image, and also shows an organ and a lesion detected by each of the detectors M1 to M7. The detection unit 32 detects regions of an organ and a lesion as regions of interest from the medical image by applying each of the detectors M1 to M7 shown in FIG. 6 to the medical image acquired by the acquisition unit 30. For example, by applying the detector M1 to the medical image of the lung shown in FIG. 3, the detection unit 32 detects a region SA of a structure showing the lung and a region AA of an abnormal shadow due to a lung nodule as regions of interest. In addition, in a case where there are a plurality of medical images acquired by the acquisition unit 30, the detection unit 32 applies each of the detectors M1 to M7 to each medical image.

As the detector, for example, a trained model such as convolutional neural network (CNN), which has been trained in advance so that the input is a medical image and the output is the region of interest detected from the medical image, may be used. This trained model is, for example, a model trained by machine learning using a large number of medical images in which a region of interest, that is, a region having a predetermined physical feature, is known, as training data. The “region having a physical feature” includes, for example, a region in a range in which the pixel value is preset (for example, a region in which the pixel value is relatively white/black mass as compared with the surroundings) and a region having a preset shape. That is, the detector may be prepared for each combination of the organ and the physical feature.

The method of detecting the region of interest by the detection unit 32 is not limited to the detection method using the detector, and for example, a region in the medical image designated by the user via the input unit 25 may be detected as the region of interest.

Further, the detection unit 32 generates element information based on the medical image acquired by the acquisition unit 30. The element information generated by the detection unit 32 may be information related to a plurality of types of structures or may be information related to a plurality of types of abnormal shadows. For the generation of the element information by the detection unit 32, for example, a trained model such as CNN, which has been trained in advance so that the input is the region of interest detected from the medical image and the output is the element information related to the region of interest, may be used.

For example, the detection unit 32 may detect a region of the abnormal shadow included in the medical image acquired by the acquisition unit 30 as a region of interest and generate element information related to the detected region of the abnormal shadow. Further, for example, the detection unit 32 may extract a region of the structure included in the medical image acquired by the acquisition unit 30 as a region of interest, extract a region of the abnormal shadow from the extracted region of the structure, and generate element information related to the extracted region of the abnormal shadow.

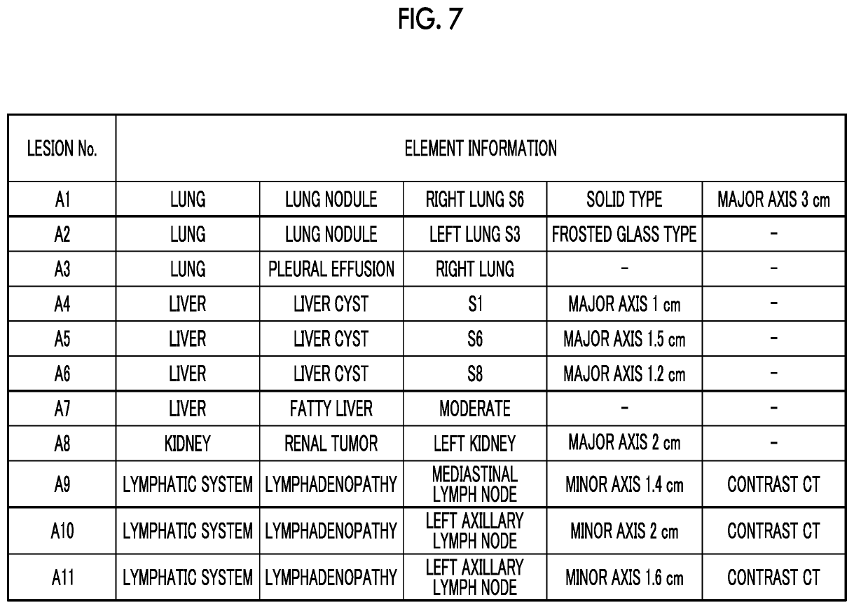

FIG. 7 shows an example of element information generated by the detection unit 32. FIG. 7 is a list of element information for each of the lesions A1 to A11 (that is, the region AA of the abnormal shadow) detected from the medical image. As shown in FIG. 7, the element information may be, for example, information indicating at least one element such as a name (type), a property, a measured value, a position, and an estimated disease name (including a negative or positive evaluation result) related to a region of interest included in a medical image.

Examples of names (types) include the names of structures such as “lung” and “liver”, and the names of abnormal shadows such as “lung nodule” and “liver cyst”. The property mainly mean the features of abnormal shadows. For example, in the case of a lung nodule, findings indicating absorption values such as “solid type” and “frosted glass type”, margin shapes such as “clear/unclear”, “smooth/irregular”, “spicula”, “lobulation”, and “serration”, and an overall shape such as “round shape” and “irregular shape” can be mentioned. In addition, for example, there are findings regarding the relationship with surrounding tissues such as “pleural contact” and “pleural invagination”, and the presence or absence of contrast enhancement, washout, and the like.

Examples of the measured value include a value that can be quantitatively measured from a medical image, and examples thereof include a major axis, a minor axis, a volume, a CT value whose unit is HU, the number of regions of interest in a case where there are a plurality of regions of interest, and a distance between regions of interest. Further, the measured value may be replaced with a qualitative expression such as “large/small” or “more/less”. The position means an anatomical position, a position in a medical image, and a relative positional relationship with other regions of interest such as “inside”, “margin”, and “periphery”. The anatomical position may be indicated by an organ name such as “lung” and “liver”, and may be expressed in terms of subdivided lungs such as “right lung”, “upper lobe”, and apical segment (“S1”). The estimated disease name is an evaluation result estimated by the detection unit 32 based on the abnormal shadow, and, for example, the disease name such as “cancer” and “inflammation” and the evaluation result such as “negative/positive”, “benign/malignant”, and “mild/severe” regarding disease names and properties can be mentioned.

The element information is not limited to the information generated based on the medical image, and may be any information that can be acquired by the detection unit 32. For example, the detection unit 32 may generate element information based on the information input via the input unit 25. Specifically, the detection unit 32 may generate element information based on the keywords input by the user via the input unit 25. Further, for example, the detection unit 32 may present a candidate for element information on the display 24 and receive the designation of the element information by the user.

Further, as described above, each medical image is attached by accessory information including information related to imaging at the time of being registered in the image DB 6. Therefore, for example, the detection unit 32 may generate, as element information, information indicating at least one of an imaging method, an imaging condition, or an imaging date and time related to the imaging of the medical image based on the accessory information attached to the medical image acquired from the image server 5.

Further, for example, the detection unit 32 may acquire element information generated in advance by an external device having a function of generating element information based on a medical image as described above from the external device. Further, for example, the detection unit 32 may acquire various types of information included in an examination order and an electronic medical record, information indicating various test results such as a blood test and an infectious disease test, information indicating the result of a health diagnosis, and the like from the external device such as the medical care WS 4, and generate the acquired information as element information as appropriate.

That is, the detection unit 32 may acquire a plurality of pieces of element information related to a medical image by generating element information based on at least one of the medical image, information input via the input unit 25, or accessory information, acquiring element information from an external device, or the like. In addition, in a case where a plurality of regions of interest are included in one medical image, the detection unit 32 may acquire element information related to each of the plurality of regions of interest included in the medical image. In addition, in a case where there are a plurality of medical images for which an interpretation report is to be created, the detection unit 32 may acquire element information related to each of the plurality of medical images. In the following description, in a case where the detection unit 32 “acquires” the element information, the element information to be acquired includes the element information generated by the detection unit 32.

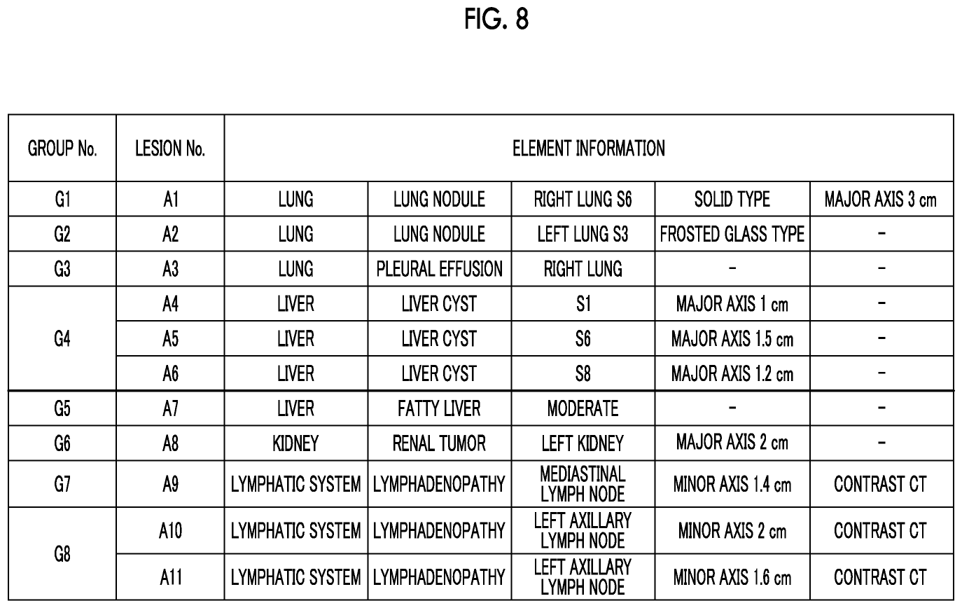

The generation unit 34 divides a plurality of pieces of element information acquired by the detection unit 32 into groups. Specifically, the generation unit 34 divides the plurality of pieces of element information into groups according to a predetermined rule. FIG. 8 shows an example in which the element information for each of the lesions A1 to A11 shown in FIG. 7 is divided into groups G1 to G8.

The generation unit 34 may divide the plurality of pieces of element information into groups for each of the regions of interest. That is, the generation unit 34 may divide the plurality of pieces of element information into groups for each of regions of abnormal shadows such as lesions, or may divide the element information into groups for each of regions of structures such as organs. In the example of FIG. 8, the generation unit 34 divides element information related to each of the lesions A1 to A3 (that is, the regions of the abnormal shadow as the regions of interest) into groups G1 to G3.

In addition, the generation unit 34 may collect element information related to each of two or more similar regions of interest among the plurality of regions of interest in the same group. The “similar” means, for example, satisfying at least one of a case where the names (types) of the regions of interest are the same, a case where the properties are the same, or a case where the measured values are within a predetermined range. Specifically, in a case where the sizes of the plurality of lesions are within a predetermined range, a case where a difference between the sizes of the plurality of lesions is within a predetermined range, and a case where a distance between the plurality of lesions is within a predetermined range, the plurality of lesions may be expressed to be “similar”.

Whether or not the regions of interest are similar to each other can be determined, for example, depending on whether or not the same or similar element information is included in at least one piece of element information associated with each of the regions of interest. In the example of FIG. 8, since the element information of the “liver” and the “liver cyst” among the element information associated with each of the lesions A4 to A6 is the same, the generation unit 34 determines that the lesions A4 to A6 are similar to each other and collects the element information related to the lesions A4 to A6 in one group G4.

Further, for example, it may be determined whether or not the regions of interest are similar to each other based on the degree of similarity of the image feature amounts of the regions of interest. Specifically, in a case where the center-to-center distance of the region of interest is within a predetermined range, and at least one of the circularity of the region of interest, the average value and the dispersion value of the pixel values, the major axis, the minor axis, the volume, or the like is similar, it may be determined that the regions of interest are similar to each other.

In addition, the generation unit 34 may change the grouping rule for each type of region of interest. For example, as shown in FIG. 8, the element information related to the “lung nodule” of the “lung” may be divided into groups for each lesion, and the element information related to the “liver cyst” of the “liver” may collect all lesions in one group. Further, for example, the element information related to the “liver” and the “kidney” may be collected in one group, or the element information to be collected in the same group may be predetermined. In addition, for example, the element information related to the “lung nodule” and the “pleural effusion” may be divided into different groups, and element information to be divided (that is, element information that is not collected in the same group) may be predetermined.

The above-described grouping rule may be stored in advance in, for example, the storage unit 22. In addition, the generation unit 34 may allow the user to select which of the above-described grouping rules is applied. In addition, the generation unit 34 may display the result of the grouping on the display 24 at a time when the grouping is completed and receive the editing of the grouping by the user.

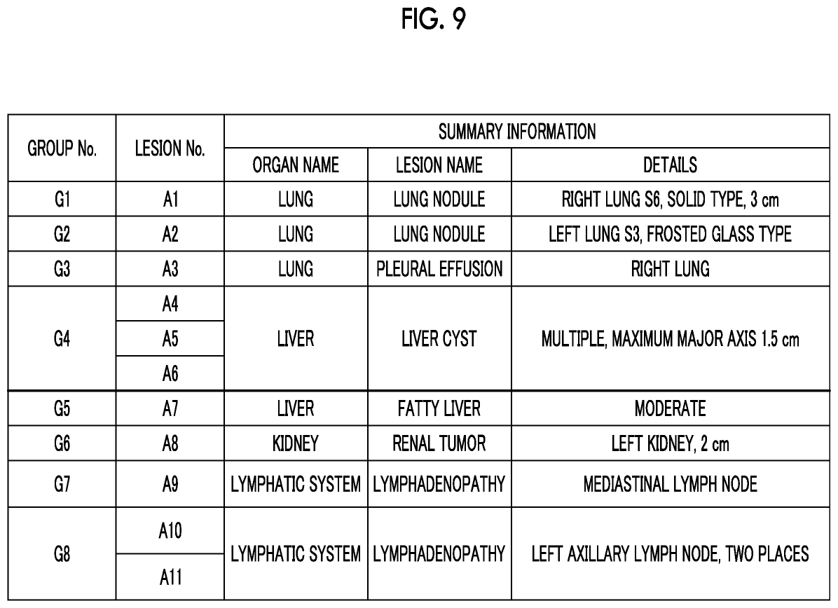

After dividing the plurality of pieces of element information into groups, the generation unit 34 generates summary information in which a summary of the element information included in each group is associated with the group. Specifically, the generation unit 34 generates summary information based on at least one piece of element information included in each group according to a predetermined rule. The summary information is generated including element information particularly important in diagnosis among element information such as an organ name, a lesion name, an anatomical position, a size, a number, and a degree of malignancy of a region of interest, for example. FIG. 9 shows an example of summary information for each of the groups G1 to G8 shown in FIG. 8.

The generation unit 34 may generate summary information based on the element information common to each of the regions of interest collected in the group. In the example of FIG. 9, the generation unit 34 generates summary information of the group G4 based on the element information indicating the organ name “liver” and the element information indicating the lesion name “liver cyst” common to each of the lesions A4 to A6 collected in the group G4.

In addition, the generation unit 34 may generate summary information based on the element information indicating at least one of the size or the position of the region of interest, which is common to each of the regions of interest collected in the group. In the example of FIG. 9, the generation unit 34 generates summary information in the “details” field of the group G8 based on the element information indicating the anatomical position of the “left axillary lymph node” common to each of the lesions A10 to A11 collected in the group G8.

In addition, the generation unit 34 may generate summary information based on the element information having the highest degree of malignancy among the element information related to each of the regions of interest collected in the group. The degree of malignancy is predetermined, for example, by the size of the lesion, the absorption value, the margin shape, the overall shape, and the like. For example, in the case of a lung nodule, the larger the size, the higher the degree of malignancy, the more solid the absorption value, the higher the degree of malignancy, and the more the margin shape and the overall shape are unclear and irregular, the higher the degree of malignancy. In the example of FIG. 9, the generation unit 34 generates summary information in the “details” field of the group G4 based on the element information indicating a size of “major axis 1.5 cm” for the lesion A5 having the largest major axis and estimated to have the highest degree of malignancy among the lesions A4 to A6 collected in the group G4.

In addition, the generation unit 34 may generate summary information based on the number of regions of interest collected in the group. In the example of FIG. 9, the generation unit 34 generates summary information of “multiple” based on the number of lesions A4 to A6 collected in the group G4. In addition, the generation unit 34 generates summary information of “two places” based on the number of the lesions A10 to A11 collected in the group G8.

Specifically, in a case where only one lesion is included in the group, it is preferable that the generation unit 34 generates information indicating the lesion name and the detailed features of the lesion as summary information (see groups G1 to G3 in FIG. 9). In a case where a plurality of lesions of the same type are included in the group, it is preferable that the generation unit 34 generates information indicating the number of regions of interest, such as “multiple” and “two”, as summary information (see groups G4 and G8 in FIG. 9).

In a case where the group includes two types of lesions related to the same organ, it is preferable that the generation unit 34 generates each lesion name as summary information and information indicating the relative positional relationship of the two types of lesions such as “mixed”, “inclusion”, and “contact”. In a case where the group includes three or more types of lesions related to the same organ, the generation unit 34 may generate information in which at least a part of each lesion name such as “multiple disease”, “lung nodule and pleural effusion, etc.”, and “organ abnormality” is omitted as summary information. This is because, in a case where the amount of information per group is large, the readability can be improved by omitting some information.

In a case where the group includes lesions related to different organs and the relationship between the primary and the metastasis is suspected, it is preferable that the generation unit 34 generates information indicating the relationship between the primary and the metastasis as the summary information.

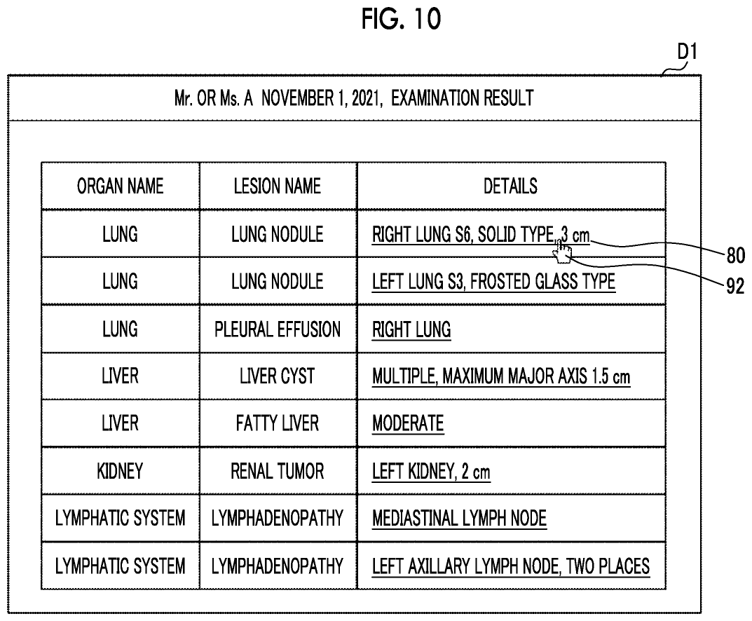

The controller 36 performs control to display the summary information for each group generated by the generation unit 34 on the display 24 in a tabular format. FIG. 10 shows an example of a screen D1 in which summary information for each group is displayed in a tabular format, which is displayed on the display 24 by the controller 36.

In addition, the controller 36 may perform control to set at least one of the medical images related to the element information included in the group as a representative image, and to display the summary information for each group and the representative image on the display 24 in association with each other. For example, the controller 36 may perform control to set a medical image from which the element information is detected as a representative image, to assign a hyperlink 80 to the representative image to the summary information, and to display the representative image on the display 24 in a case where the hyperlink 80 is selected.

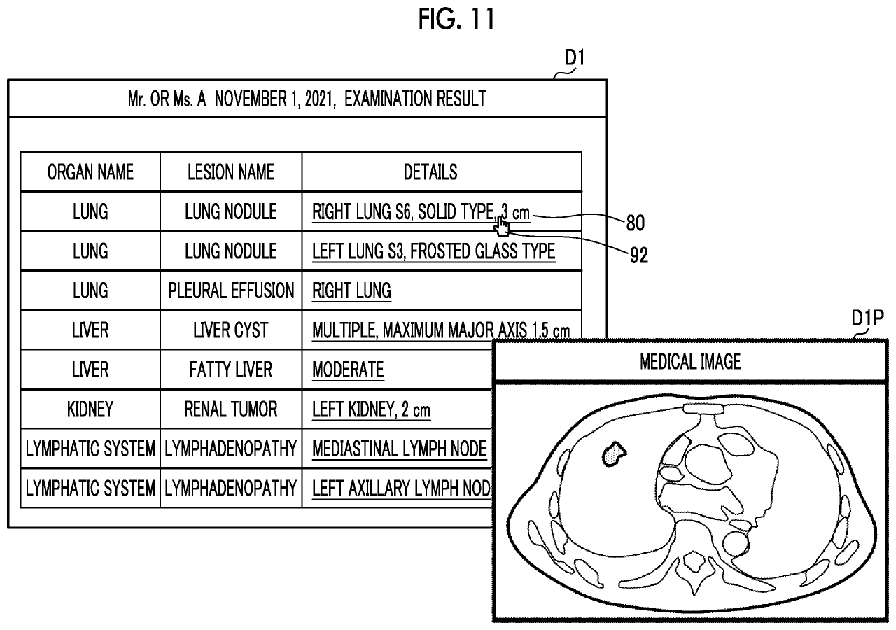

In the example of FIG. 10, a hyperlink 80 to at least one representative image set for each group is added to the character string indicating the summary information in the “details” field. The user operates a cursor 92 on the screen D1 via the input unit 25, and selects the summary information in a case where he/she desires to view the medical image, thereby making a viewing request. For example, in a case where the hyperlink 80 added to the character string indicating the summary information “right lung S6, solid type, 3 cm” is selected on the screen D1 of FIG. 10, the controller 36 performs control to display a pop-up window D1P including the corresponding representative image on the display 24 as shown in FIG. 11. According to such a form, since the medical image is displayed on the display 24 after receiving the viewing request by the user, the amount of information initially displayed can be reduced to only the summary information, and the visibility for the user can be improved.

The controller 36 may extract a partial region including the region of interest from the medical image from which the element information is acquired, and set the partial region as the representative image.

In addition, in a case where element information related to a plurality of regions of interest is collected in the group, the controller 36 may set the following various images as representative images. First, the controller 36 may set a medical image from which the element information having the highest degree of malignancy is acquired as a representative image. For example, the controller 36 may set a medical image from which a lesion estimated to have the largest major axis and the highest degree of malignancy is detected as a representative image.

Second, the controller 36 may set all the medical images from which the plurality of regions of interest included in the group are acquired as representative images. For example, in a case where three lesions are included in a group, the controller 36 may set three medical images in which each lesion is detected as a representative image.

Third, the controller 36 may generate one or a plurality of reconstructed images including all of the plurality of regions of interest included in the group based on the plurality of tomographic images T1 to Tm and set the generated reconstructed images as representative images. The reconstructed image is, for example, an image generated by a method such as multi-planer reconstruction (MPR), curved-planer reconstruction (CPR), volume rendering (VR), or maximum intensity projection (MIP). Further, for example, an image reconstructed by changing a window level (WL), a window width (WW), a field of view (FOV), or the like in the tomographic image may be set as the representative image.

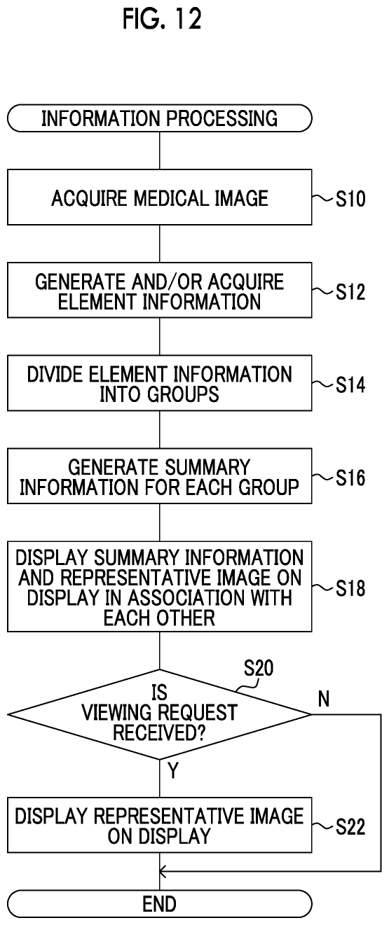

Next, with reference to FIG. 12, operations of the information processing apparatus 10 according to the present embodiment will be described. In the information processing apparatus 10, the CPU 21 executes the information processing program 27, and thus information processing shown in FIG. 12 is executed. The information processing is executed, for example, in a case where the user gives an instruction to start execution via the input unit 25.

In Step S10, the acquisition unit 30 acquires a medical image from the image server 5. In Step S12, the detection unit 32 detects a region of interest included in the medical image acquired in Step S10 and generates element information. In addition, the detection unit 32 may generate element information based on information input by the user via the input unit 25 and information acquired from an external device, or may acquire element information from an external device. In Step S14, the generation unit 34 divides a plurality of pieces of element information generated and/or acquired in Step S12 into groups. In Step S16, the generation unit 34 generates summary information in which a summary of element information included in each group divided in Step S14 is associated with the group.

In Step S18, the controller 36 performs control to display the summary information for each group on the display 24. Specifically, the controller 36 sets at least one of the medical images related to the element information included in the group as a representative image, and displays the summary information for each group and the representative image on the display 24 in association with each other. In Step S20, the controller 36 waits until a viewing request for the representative image by the user is received. In a case where the viewing request is received by the user (that is, in a case where Step S20 is Y), the process proceeds to Step S22, and the controller 36 performs control to display the representative image for which the viewing request has been received on the display 24, and ends the present information processing. On the other hand, in a case where there is no viewing request by the user (that is, in a case where Step S20 is N), the process of Step S22 is not performed, and the present information processing is ended.

As described above, the information processing apparatus 10 according to one aspect of the present disclosure includes at least one processor, in which the processor acquires a plurality of pieces of element information related to a medical image, divides the plurality of pieces of element information into groups, and generates summary information in which a summary of the element information included in the group is associated with the group. That is, with the information processing apparatus 10 according to the present embodiment, even though a large amount of element information can be obtained from the medical image, the overall overview can be easily viewed by the summary information, and thus it is possible to support the creation of an interpretation report.

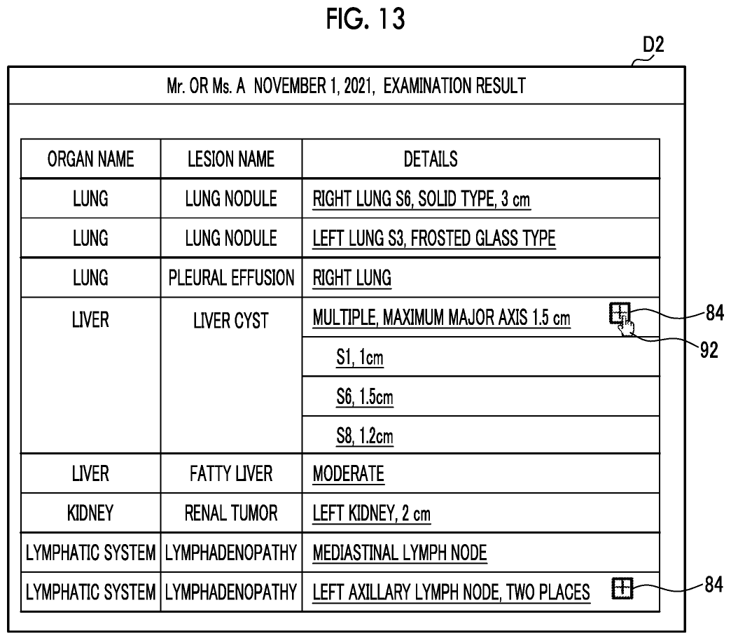

Hereinafter, each modification example of the above-described embodiment will be described with reference to FIGS. 13 to 27. Each of the following modification examples can be appropriately combined.

### Modification Example 1

It is expected that a user who has viewed the summary information for each group by the technique according to the embodiment may subsequently desire to view the detailed information of the lesion included in the group. Therefore, the information processing apparatus 10 according to Modification Example 1 may present more detailed summary information in a case where a viewing request is received from the user.

Specifically, in a case where a plurality of lesions are included in the group, the generation unit 34 may generate both the summary information of the group and the summary information for each lesion included in the group. The controller 36 may initially display only the summary information of the group on the display 24, and also display the summary information for each lesion on the display 24 in a case where there is a viewing request from the user.

FIG. 13 is a diagram showing an example of a screen D2 displayed on the display 24 by the controller 36, and is a modification example of the screen D1 of FIG. 10. In FIG. 13, an expand button 84 is displayed in a field of “multiple, maximum major axis 1.5 cm” and a field of “left axillary lymph node, two places”. On the screen D2, the field of “multiple, maximum major axis 1.5 cm” is in a state in which the summary information is expanded and the summary information for each lesion is displayed, and the field of “left axillary lymph node, two places” is in a state in which the summary information is aggregated without being expanded.

The user operates the cursor 92 on the screen D2 via the input unit 25, and selects the expand button 84 in a case of requesting viewing of the summary information for each lesion. In a case where the expand button 84 is selected, the controller 36 performs control to also display the summary information for each lesion on the display 24. By presenting the summary information step by step in this way, it is possible to easily view the overall overview at first, and at the same time, it is possible to view detailed information as desired by the user, and it is possible to support the creation of an interpretation report.

The means for presenting the detailed summary information is not limited to the expand button 84. For example, the controller 36 may give the same function as the expand button 84 to the character string indicating the summary information in the “details” field on the screen D2, instead of the hyperlink 80 to the representative image.

### Modification Example 2-1

In the above-described embodiment, the controller 36 may receive status information indicating the status of the work performed with regard to the element information collected in the group for each group and assign the status information to the group. The status information is, for example, a status indicating whether or not the user has checked the lesion, a status indicating whether or not the lesion has been described in the interpretation report, and the like. In addition, the status information is, for example, a status in which the user determines that a lesion detected by the detection unit 32 is an erroneous detection, a status in which the user determines that a lesion detected by the detection unit 32 is not described in the interpretation report, and the like.

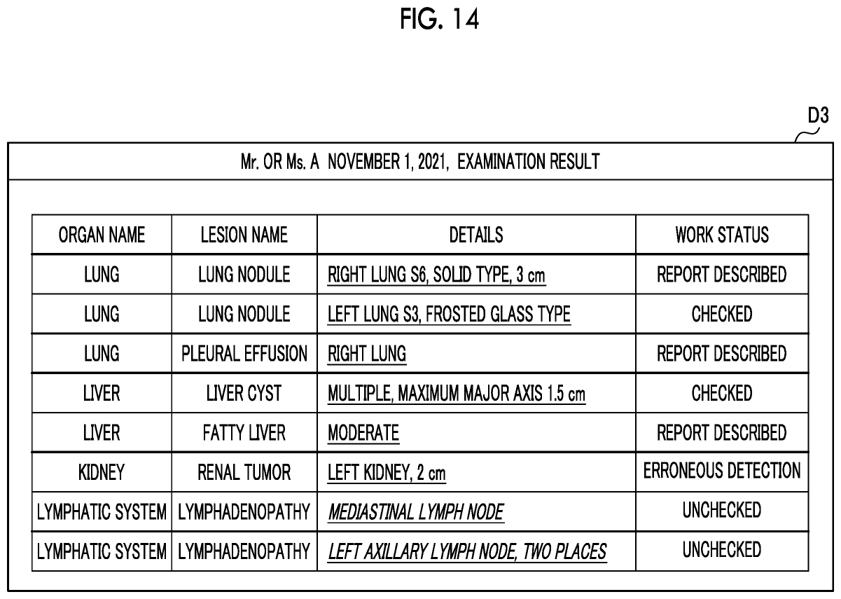

In addition, the controller 36 may perform control to display the summary information for each group and the status information assigned to the group on the display 24 in association with each other. FIG. 14 is a diagram showing an example of a screen D3 provided with a field of “work status” corresponding to summary information (fields of “organ name”, “lesion name”, and “details”), which is displayed on the display 24 by the controller 36. In addition, the controller 36 may receive the setting of the status information by the user on the screen D3. By making the status information viewable in this way, the user can easily grasp the progress of the work and specify the information to be confirmed with priority, and thus it is possible to support the creation of an interpretation report.

In addition, the controller 36 may change the display form of the summary information according to the status information. Examples of the method of changing the display form include different character colors, thicknesses, italic, and font types, different character background colors, and different line types of character enclosing lines. In the example of FIG. 14, the characters of the summary information (in the field of “details”) to which the status information of “unchecked” is assigned are in bold or italic, and are highlighted more than the summary information to which other status information is assigned. According to such a form, it is possible to further easily specify the information to be confirmed with priority, and it is possible to improve the visibility for the user.

### Modification Example 2-2

In Modification Example 2-1 above, in a case where the status information indicating that the group has already been described in the interpretation report is assigned, the controller 36 may perform control to acquire an interpretation report describing the group to which the status information is assigned, and to display at least a part of the interpretation report on the display 24. In this case, the controller 36 may perform control to display at least a part of the interpretation report on the display 24 in a case where a viewing request for the interpretation report is received from the user.

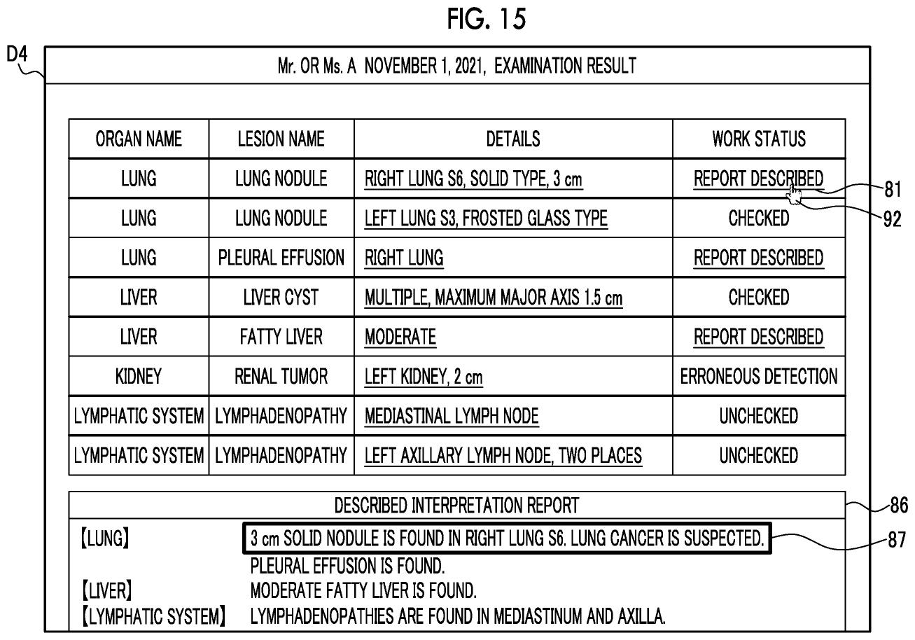

FIG. 15 is a diagram showing an example of a screen D4 provided with a field of “work status” corresponding to summary information (fields of “organ name”, “lesion name”, and “details”) and further including a completed interpretation report 86, which is displayed on the display 24 by the controller 36. On the screen D4, in a case where the status information of “report described” is selected, at least a part of the interpretation report 86 describing the group to which the status information is assigned is highlighted by a bounding box 87. The screen D4 is a state in which a part of the interpretation report 86 describing the group in which the “details” field is “right lung S6, solid type, 3 cm” is highlighted by the bounding box 87.

In a case where the user operates the cursor 92 on the screen D4 via the input unit 25 to request viewing of the interpretation report, the user selects a character string 81 of “report described” in the “work status” field. In a case where the character string 81 of “report described” is selected, the controller 36 specifies at least a part corresponding to the selected group in the entire sentence of the interpretation report 86 based on the element information collected in the group to which the selected “report described” status information is assigned. For example, the controller 36 specifies a sentence including a word having the same meaning as the element information collected in the selected group in the entire sentence of the interpretation report 86. Then, the controller 36 surrounds the specified part of the interpretation report 86 with the bounding box 87 and highlights it.

### Modification Example 2-3

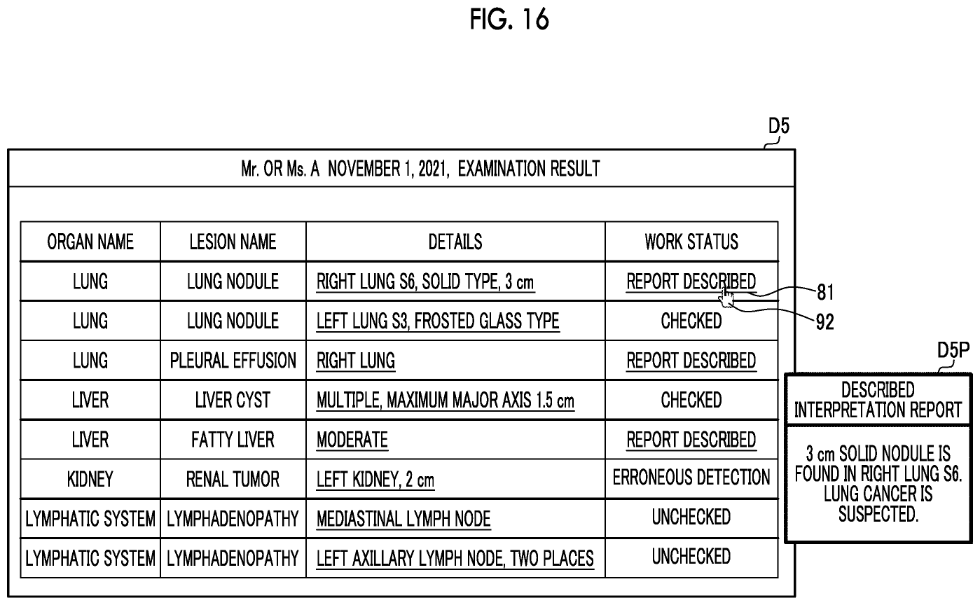

FIG. 16 is a modification example of the screen D4 of FIG. 15, and is a diagram showing an example of a screen D5 in which a part of the interpretation report 86 describing the selected group is displayed in a pop-up window D5P. As shown on the screen D5, in a case where the character string 81 of “report described” is selected, the controller 36 may display at least a part corresponding to the selected group in the entire sentence of the interpretation report 86 in the pop-up window D5P.

### Modification Example 2-4

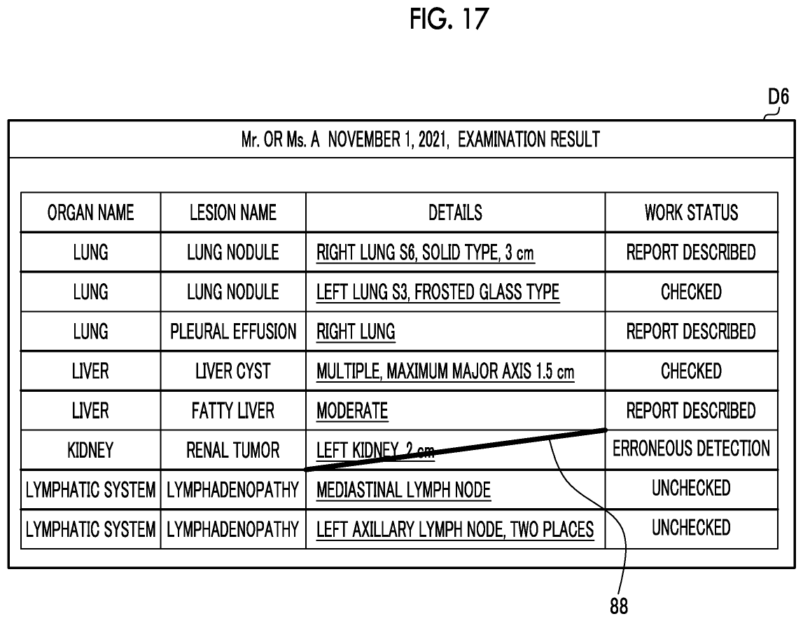

In Modification Example 2-1 above, in a case where status information indicating that the user has determined that it is an erroneous detection is assigned, the controller 36 may change a display form of the summary information of the group to which the status information is assigned. For example, FIG. 17 shows an example of a screen D6 in which a strikethrough 88 is added to summary information of a group to which the work status of an “erroneous detection” is assigned, as a modification example of the screen D3 in FIG. 14. In addition to this, for example, by different character colors, thicknesses, italic, and font types, different character background colors, and different line types of character enclosing lines, a display form of summary information of a group to which the work status of an “erroneous detection” is assigned may be changed. By displaying the information so that it is easy to understand that it is an erroneous detection, the user can gaze at or ignore the information, and thus it is possible to support the creation of an interpretation report.

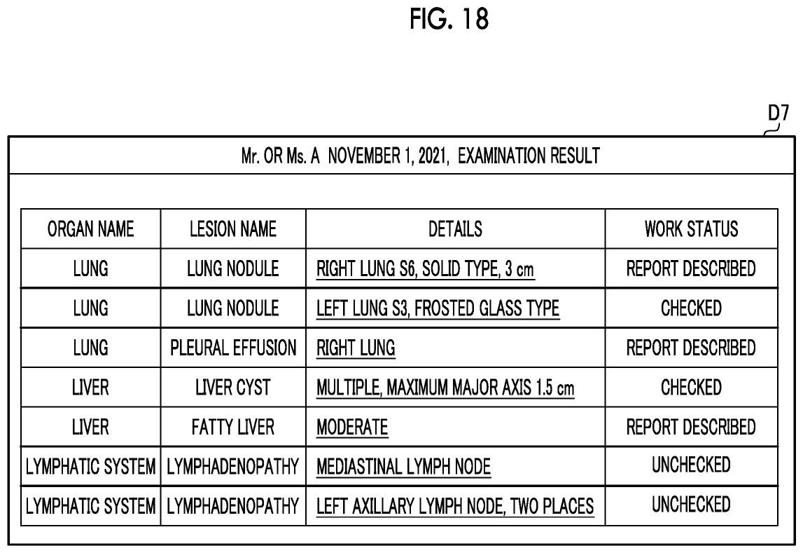

Further, for example, FIG. 18 shows an example of a screen D7 in which the summary information of the group to which the work status of an “erroneous detection” is assigned is deleted, as a modification example of the screen D3 in FIG. 14. As shown in FIG. 18, in a case where status information indicating that the user has determined that it is an erroneous detection is assigned, the controller 36 may not display the summary information of the group to which the status information is assigned.

### Modification Example 3

The information processing apparatus 10 according to Modification Example 3 may generate summary information indicating a change with time of each lesion based on medical images such as a current image and a past image, which are captured at different imaging points in time. Specifically, the acquisition unit 30 may acquire a plurality of medical images (for example, a current image and a past image) captured at different imaging points in time from the image server 5. The detection unit 32 may detect a region of interest from each of the plurality of medical images and generate element information. The generation unit 34 collects element information related to two or more medical images having the same region of interest of the same subject as an imaging target and captured at different imaging points in time in the same group. In addition, the generation unit 34 generates summary information based on a comparison result between the pieces of element information related to each of the two or more medical images collected in the same group and having the same region of interest of the same subject as an imaging target and captured at different imaging points in time.

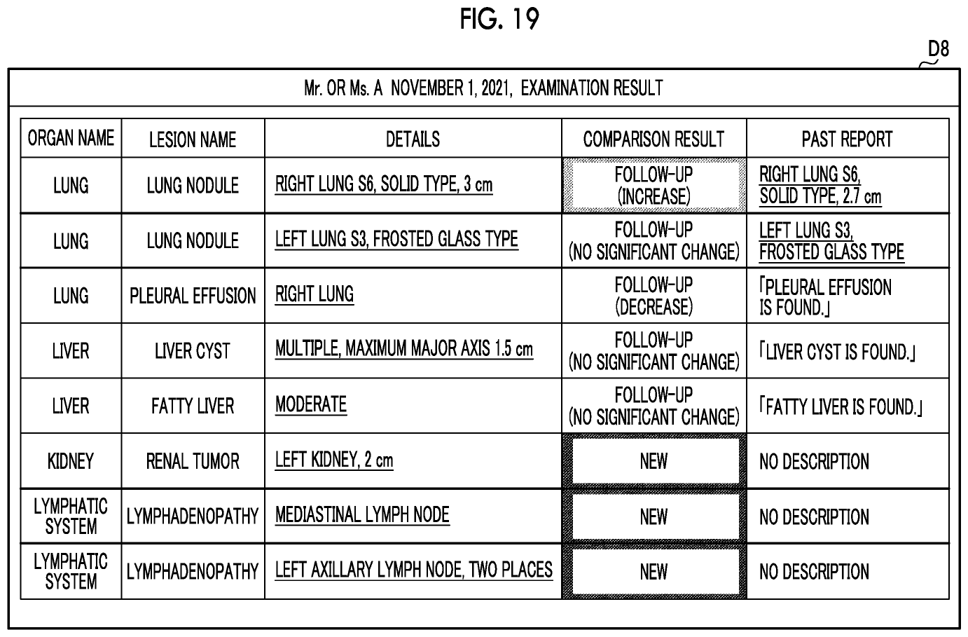

FIG. 19 is a diagram showing an example of a screen D8 including summary information (“comparison result” field) showing a comparison result with a past image and an interpretation report (“past report” field) created based on the past image, which is displayed on the display 24 by the controller 36. In a case where the element information related to the past image corresponding to the element information related to the current image is present, the generation unit 34 may determine that the element information is related to a follow-up lesion, and generate summary information indicating “follow-up” as shown in FIG. 19. On the other hand, in a case where the element information related to the past image corresponding to the element information related to the current image is not present, the generation unit 34 may determine that the element information is related to a new lesion, and generate summary information indicating “new” as shown in FIG. 19.

In addition, in a case where the element information is determined to be related to the follow-up lesion, the generation unit 34 may generate a comparison result indicating a change from element information related to a medical image (past image) captured at an earlier imaging point in time to element information related to a medical image (current image) captured at a later imaging point in time. The “comparison result showing a change” is, for example, improvement or deterioration of properties, enlargement or reduction of lesion size, occurrence or disappearance of lesion, degree of these changes (large/small/no change), and the like. FIG. 19 illustrates the summary information of “increase”, “decrease”, and “no significant change” as the comparison result showing the change.

In a case where the summary information for each group is displayed on the display 24, the controller 36 may change the display form according to the comparison result generated by the generation unit 34. Examples of the method of changing the display form include different character colors, thicknesses, italic, and font types, different character background colors, and different line types of character enclosing lines. For example, as shown in FIG. 19, among the summary information indicating “follow-up”, the background color of the “increase (that is, deterioration) summary information may be highlighted by making it different from the background color of the “decrease” and “no significant change” (that is, improvement and no change) summary information. Further, for example, as shown in FIG. 19, the background color of the summary information may be changed between “follow-up” and “new”.

Further, for example, as shown in “right lung S6, solid type, 2.7 cm” in the “past report” field of FIG. 19, the generation unit 34 may generate summary information related to the past image based on the element information related to the past image, and the controller 36 may display the summary information related to the past image in association with summary information related to the current image. Further, for example, as shown in “‘pleural effusion is found’” in the “past report” field of FIG. 19, the controller 36 may acquire a past interpretation report (hereinafter referred to as a “past report”) created based on the past image and specify at least a corresponding part in the entire sentence of the past report for each group and display the corresponding part in association with the summary information. Further, for example, in a case where the character string of “right lung S6, solid type, 2.7 cm” in the “past report” field of FIG. 19 is selected, the controller 36 may display at least a part corresponding to the selected group in the entire sentence of the past report in the pop-up window.

### Modification Example 4-1

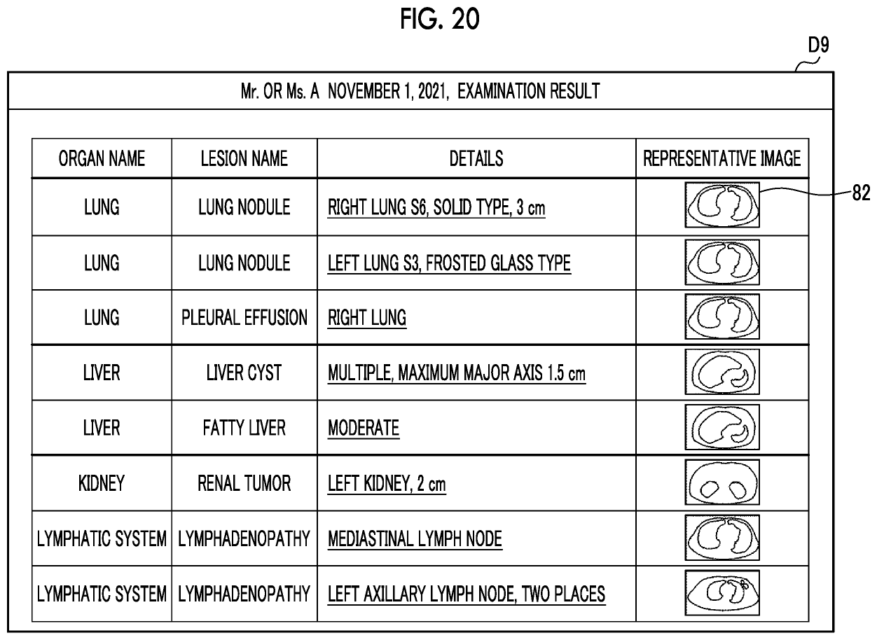

In the above-described embodiment, the form in which the representative image set for each group is associated with the summary information by a hyperlink and displayed on the display 24 (see FIGS. 10 and 11) has been described. However, the form of association between the representative image and summary information is not limited thereto. FIG. 20 is a diagram showing an example of a screen D9 on which thumbnail images 82 of representative images are displayed in a tabular format in association with summary information, which is displayed on the display 24 by the controller 36. As shown in FIG. 20, in a case where a thumbnail image 82 based on a representative image is created and the summary information is displayed in a tabular format on the display 24, the controller 36 may also incorporate the thumbnail image 82 into the table.

### Modification Example 4-2

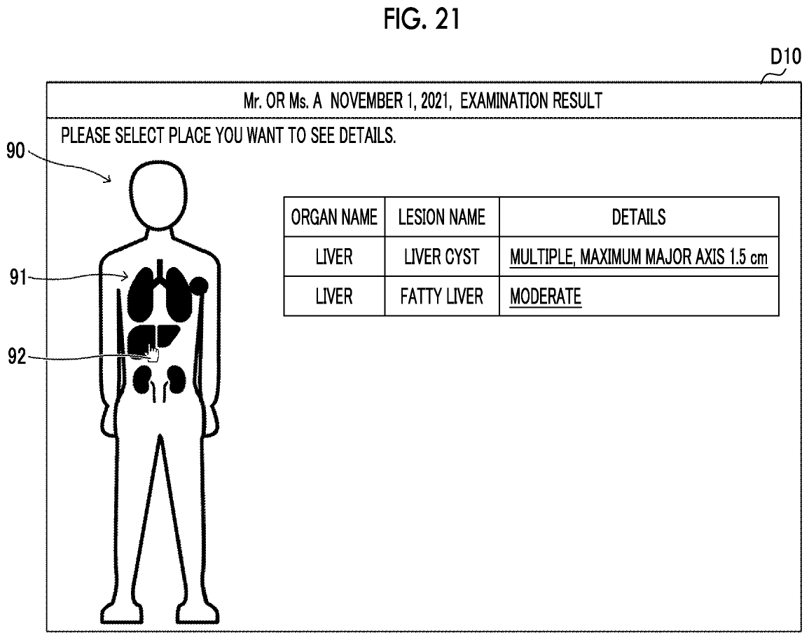

The controller 36 may perform control to assign summary information to regions corresponding to each group on a schema showing a human body and display the summary information on the display 24. FIG. 21 is a diagram showing an example of a screen D10 on which a schema 90 showing a human body and summary information are associated with each other, which is displayed on the display 24 by the controller 36. In FIG. 21, an icon 91 of an organ (lung, liver, kidney, or lymphatic system) corresponding to each piece of the element information indicating the organ acquired by the detection unit 32 is displayed on the schema 90 showing the human body. FIG. 21 shows a state in which the liver icon 91 is selected and summary information related to the liver is displayed.

The user operates the cursor 92 on the screen D10 via the input unit 25 to select the icon 91 of the organ for which viewing of the summary information is requested. In a case where the icon 91 is selected, the controller 36 performs control to display the summary information corresponding to the selected organ on the display 24. By presenting the summary information using the schema in this way, it is possible to easily view at which position each lesion occurs, and it is possible to support the creation of an interpretation report.

In addition, the controller 36 may change the display form of the icon 91 based on the summary information indicating the comparison result with the past image described in Modification Example 3 above. For example, the controller 36 may change the color of the icon 91 between the follow-up lesion and the new lesion.

### Modification Example 5

It is expected that a user who has viewed the representative image for each group by the technique according to the embodiment may subsequently desire to view another medical image that is not set as the representative image. Therefore, the information processing apparatus 10 according to Modification Example 5 may receive the selection of the medical image to be displayed by the user.

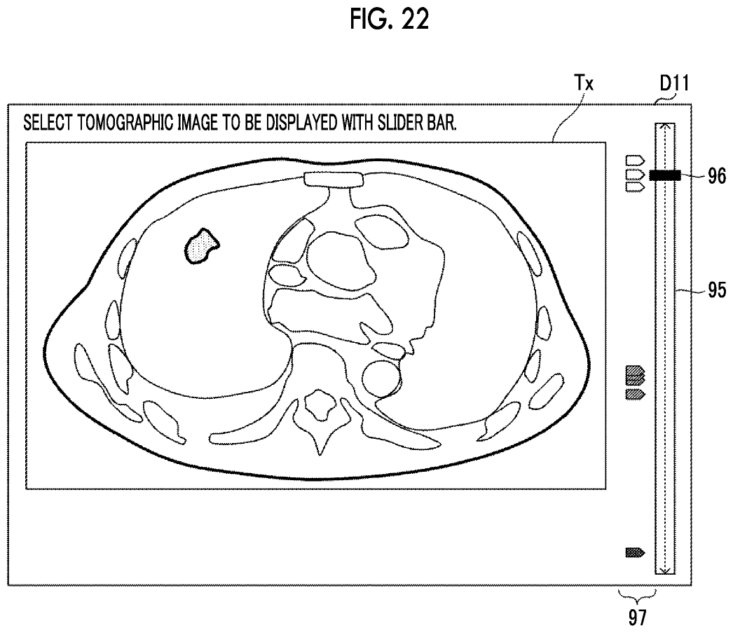

FIG. 22 shows an example of a screen D11 for selecting a medical image to be displayed on the display 24. The screen D11 is a screen displayed on the display 24 by the controller 36 in a case where a character string indicating the summary information in the “details” field is selected instead of the pop-up window D1P including the representative image as shown in FIG. 11, for example.

The screen D11 includes a slider bar 95 for receiving an operation of selecting an image to be displayed on the display 24 from a plurality of tomographic images T1 to Tm (see FIG. 2). The slider bar 95 is a graphical user interface (GUI) part that is also called a slide bar or a scroll bar. An example of the screen D11 corresponds to a plurality of tomographic images T1 to Tm arranged in order from the chest side to the lumbar side from the upper end to the lower end.

The controller 36 receives an operation of the position of a slider 96 on the slider bar 95 by the user via the input unit 25, and displays, on the screen D11, one image (the tomographic image Tx in the example of FIG. 22) corresponding to the position of the slider 96 among the plurality of tomographic images T1 to Tm. The dotted arrow added to the slider 96 in FIG. 22 means the movable range of the slider 96 in the slider bar 95.

In addition, the controller 36 may display markers 97 having different forms according to the element information generated based on the respective tomographic images T1 to Tm at the corresponding positions of the slider bar 95. The screen D11 of FIG. 22 includes markers 97 having different forms disposed on the side of the slider bar 95. The marker 97 is for indicating a position on the slider bar 95 of the image in which the lesion is detected among the plurality of tomographic images T1 to Tm. The form of the marker 97 may be determined according to the element information detected based on the tomographic image, or may be color-coded for each type of the detected organ, for example.

In addition, the controller 36 may receive the selection of the marker 97 to be displayed on the screen D11. For example, the controller 36 may perform control to receive the designation of the type of the organ and/or the lesion and to display only the marker 97 corresponding to the tomographic image including the designated organ and/or lesion on the screen D11.

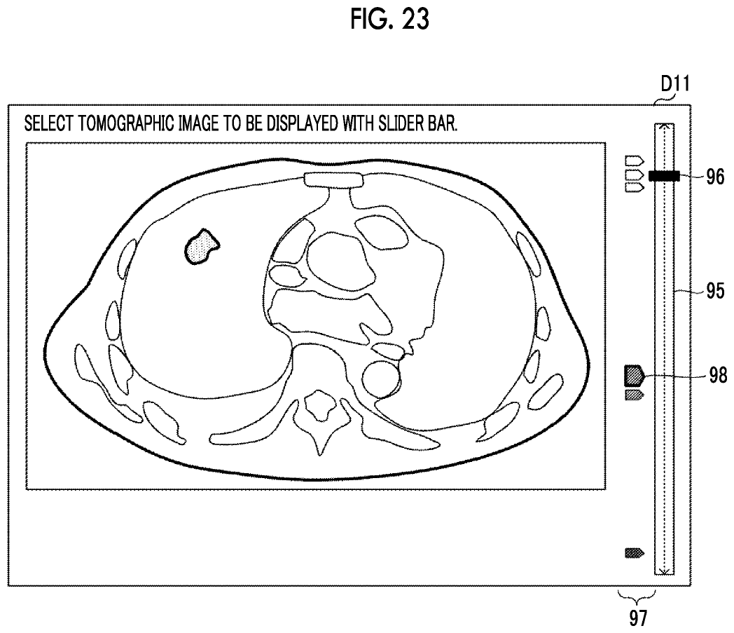

In addition, the controller 36 may also collect the markers 97 in a group as the element information is collected in the group. For example, as shown in lesions A4 to A6 in FIG. 9, in a case where a plurality of lesions are collected in the group, three markers 97 corresponding to the tomographic images from which the lesions A4 to A6 are detected may be collected in one integrated marker 98. FIG. 23 shows an example of a screen D12 in which three markers 97 are replaced with one integrated marker 98 as a modification example of the screen D11 of FIG. 22.

### Modification Example 6-1

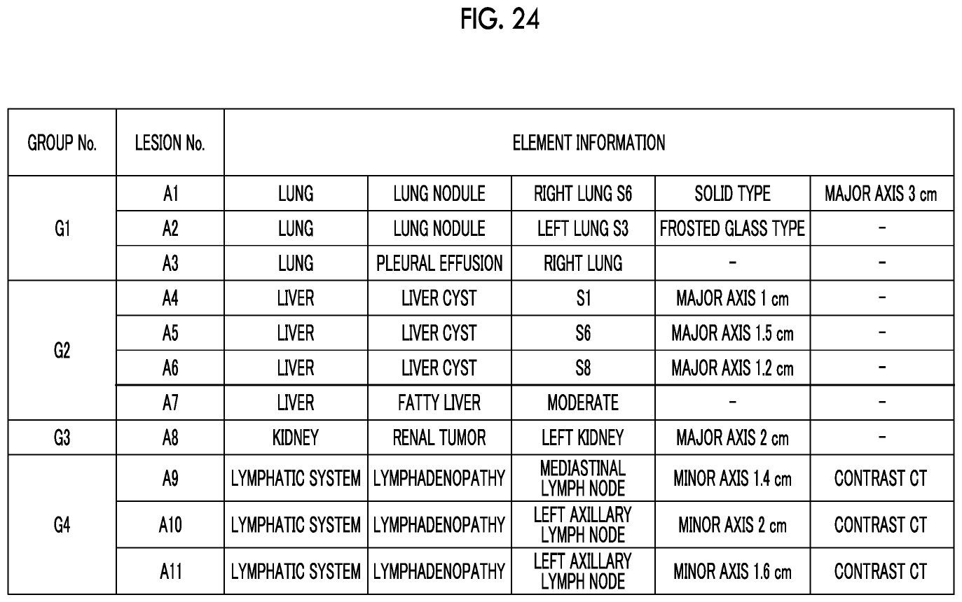

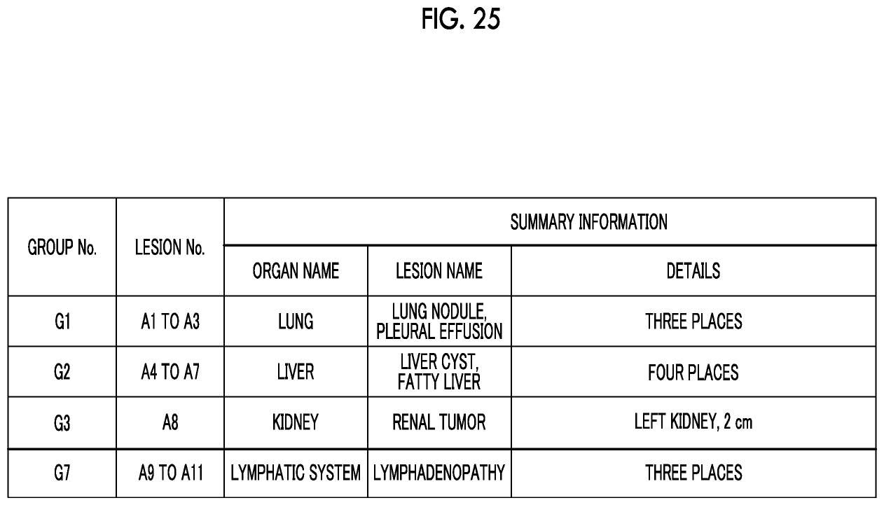

In Modification Examples 6-1 to 6-5, an example of a grouping rule different from the above-described embodiment will be described. As shown in FIG. 24, the generation unit 34 may group a plurality of pieces of element information for each organ (“lung”, “liver”, “kidney”, and “lymphatic system”). An example of the summary information in this case is shown in FIG. 25.

### Modification Example 6-2

The generation unit 34 may divide the plurality of pieces of element information into groups for each medical image. That is, the generation unit 34 may divide the plurality of pieces of element information into groups for each medical image of the acquisition source. For example, in a case where a plurality of pieces of element information of “lung nodule” and “lymphadenopathy” are acquired from one medical image, these pieces of element information may be collected in one group.

### Modification Example 6-3

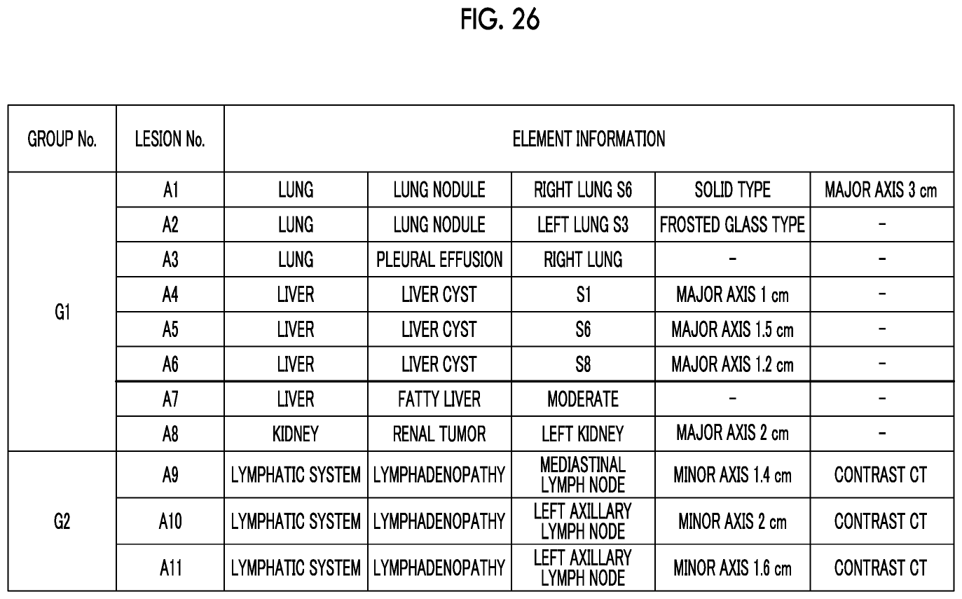

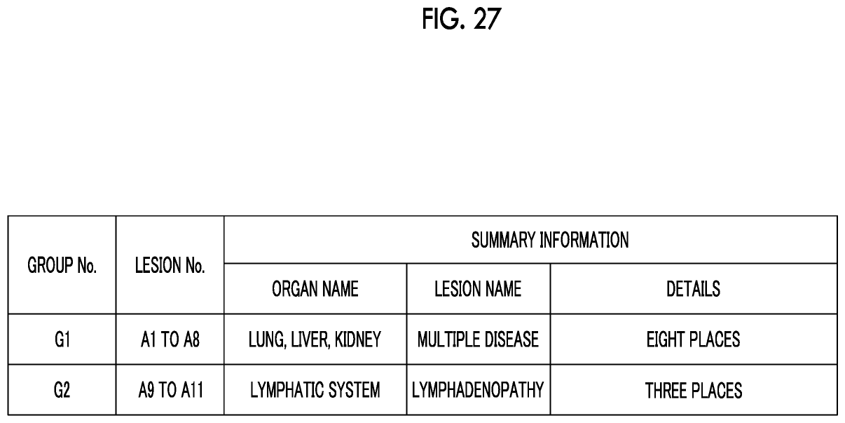

In Modification Example 6-2 described above, the generation unit 34 may collect element information related to each of two or more medical images having at least one of the same imaging method or imaging condition among the plurality of medical images in the same group. That is, in a case where two or more medical images from which the plurality of pieces of element information is acquired are acquired under the same imaging method and/or imaging condition, the generation unit 34 may collect the element information acquired from the two or more medical images in one group. For example, as shown in FIG. 26, in a case where a plurality of pieces of element information acquired from a plurality of medical images acquired in each of the simple CT imaging and the contrast CT imaging are grouped, the group may be divided into a group G1 for simple CT imaging and a group G2 for contrast CT imaging. An example of the summary information in this case is shown in FIG. 27.

### Modification Example 6-4

The image to be created for the interpretation report may include a plurality of medical images having different imaging phases (for example, arterial phase, portal vein phase, equilibrium phase, and the like) obtained in contrast imaging. In this case, the generation unit 34 may collect element information related to each of two or more medical images having the same imaging phase among the plurality of medical images in the same group. For example, the group may be divided into an arterial phase group, a portal vein phase group, and an equilibrium phase group. In this case, as the summary information, summary information indicating an imaging phase such as “arterial phase” may be generated.

### Modification Examples 6-5

The image to be created in the interpretation report may include images having different slice thicknesses (slice intervals) in tomographic imaging in which the same region of interest of the same subject is the imaging target. For example, an interpretation report may be created based on both images (for example, a thickness of 1 mm and a thickness of 3 mm) having different slice thicknesses for the same region of interest of the same subject. In this case, the generation unit 34 may collect element information related to each of two or more medical images having the same slice thickness among the plurality of medical images in the same group. For example, the group may be divided into a group having a slice thickness of 1 mm and a group having a slice thickness of 3 mm. In this case, as the summary information, summary information indicating the slice thickness such as “1 mm” or “thin” may be generated. The image having a thicker slice thickness may be, for example, an image obtained by adding and averaging pixel values at the same coordinate positions of a plurality of consecutive images having a thin slice thickness.

In the above embodiments, for example, as hardware structures of processing units that execute various kinds of processing, such as the acquisition unit 30, the detection unit 32, the generation unit 34, and the controller 36, various processors shown below can be used. As described above, the various processors include a programmable logic device (PLD) as a processor of which the circuit configuration can be changed after manufacture, such as a field programmable gate array (FPGA), a dedicated electrical circuit as a processor having a dedicated circuit configuration for executing specific processing such as an application specific integrated circuit (ASIC), and the like, in addition to the CPU as a general-purpose processor that functions as various processing units by executing software (program).

One processing unit may be configured by one of the various processors, or may be configured by a combination of the same or different kinds of two or more processors (for example, a combination of a plurality of FPGAs or a combination of the CPU and the FPGA). In addition, a plurality of processing units may be configured by one processor.

As an example in which a plurality of processing units are configured by one processor, first, there is a form in which one processor is configured by a combination of one or more CPUs and software as typified by a computer, such as a client or a server, and this processor functions as a plurality of processing units. Second, as represented by a system on chip (SoC) or the like, there is a form of using a processor for realizing the function of the entire system including a plurality of processing units with one integrated circuit (IC) chip. In this way, various processing units are configured by one or more of the above-described various processors as hardware structures.

Furthermore, as the hardware structure of the various processors, more specifically, an electrical circuit (circuitry) in which circuit elements such as semiconductor elements are combined can be used.

In the above embodiment, the information processing program 27 is described as being stored (installed) in the storage unit 22 in advance; however, the present disclosure is not limited thereto. The information processing program 27 may be provided in a form recorded in a recording medium such as a compact disc read only memory (CD-ROM), a digital versatile disc read only memory (DVD-ROM), and a universal serial bus (USB) memory. In addition, the information processing program 27 may be downloaded from an external device via a network. Further, the technique of the present disclosure extends to a storage medium for storing the information processing program non-transitorily in addition to the information processing program.

The technique of the present disclosure can be appropriately combined with the above-described embodiments. The described contents and illustrated contents shown above are detailed descriptions of the parts related to the technique of the present disclosure, and are merely an example of the technique of the present disclosure. For example, the above description of the configuration, function, operation, and effect is an example of the configuration, function, operation, and effect of the parts according to the technique of the present disclosure. Therefore, needless to say, unnecessary parts may be deleted, new elements may be added, or replacements may be made to the described contents and illustrated contents shown above within a range that does not deviate from the gist of the technique of the present disclosure.

What is claimed is:

1. An information processing apparatus comprising at least one processor, wherein the at least one processor is configured to:
acquire a plurality of pieces of element information related to a medical image;
divide the plurality of pieces of element information into groups; and
generate summary information in which a summary of the element information included in the group is associated with the group.

2. The information processing apparatus according to claim 1, wherein the at least one processor is configured to:
acquire the element information related to each of a plurality of regions of interest included in the medical image; and
divide the plurality of pieces of element information into groups for each of the regions of interest.

3. The information processing apparatus according to claim 2, wherein the at least one processor is configured to collect the element information related to each of two or more similar regions of interest among the plurality of regions of interest in the same group.

4. The information processing apparatus according to claim 3, wherein the at least one processor is configured to generate the summary information based on the element information common to each of the regions of interest collected in the group.

5. The information processing apparatus according to claim 4, wherein the at least one processor is configured to generate the summary information based on the element information indicating at least one of a size or a position of the region of interest, which is common to each of the regions of interest collected in the group.

6. The information processing apparatus according to claim 3, wherein the at least one processor is configured to generate the summary information based on the element information having a highest degree of malignancy among the element information related to each of the regions of interest collected in the group.

7. The information processing apparatus according to claim 3, wherein the at least one processor is configured to generate the summary information based on the number of regions of interest collected in the group.

8. The information processing apparatus according to claim 1, wherein the at least one processor is configured to acquire the element information related to each of a plurality of the medical images.

9. The information processing apparatus according to claim 8, wherein the at least one processor is configured to divide the plurality of pieces of element information into groups for each of the medical images.

10. The information processing apparatus according to claim 9, wherein the at least one processor is configured to collect the element information related to each of two or more medical images having at least one of the same imaging method or imaging condition in the same group.

11. The information processing apparatus according to claim 9, wherein:
the plurality of medical images include images captured at different imaging points in time, and
the at least one processor is configured to collect the element information related to each of the plurality of medical images having the same region of interest of the same subject as an imaging target and captured at different imaging points in time in the same group.

12. The information processing apparatus according to claim 1, wherein the at least one processor is configured to display the summary information for each group on a display in a tabular format.

13. The information processing apparatus according to claim 1, wherein the at least one processor is configured to assign the summary information to regions corresponding to each group on a schema and display the summary information on a display.

14. The information processing apparatus according to claim 1, wherein the at least one processor is configured to:
set at least one of the medical images related to the element information included in the group as a representative image; and
display the summary information for each group and the representative image on a display in association with each other.

15. The information processing apparatus according to claim 1, wherein:
status information indicating a status of a work performed with regard to the element information collected in the group is assigned to each group, and
the at least one processor is configured to display the summary information for each group and the status information assigned to the group on a display in association with each other.

16. The information processing apparatus according to claim 1, wherein the at least one processor is configured to:
acquire the medical image; and
generate the element information based on the acquired medical image.

17. The information processing apparatus according to claim 16, wherein the at least one processor is configured to:
detect a region of interest from the acquired medical image; and
generate the element information related to the detected region of interest.

18. The information processing apparatus according to claim 1, wherein:
the element information is information indicating at least one of a name, a property, a measured value, a position, or an estimated disease name related to a region of interest included in the medical image, or an imaging method, an imaging condition, or an imaging date and time related to imaging of the medical image, and
the region of interest is at least one of a region of a structure included in the medical image or a region of an abnormal shadow included in the medical image.

19. An information processing method comprising:
acquiring a plurality of pieces of element information related to a medical image;
dividing the plurality of pieces of element information into groups; and
generating summary information in which a summary of the element information included in the group is associated with the group.

20. A non-transitory computer-readable storage medium storing an information processing program causing a computer to execute a process comprising:
acquiring a plurality of pieces of element information related to a medical image;
dividing the plurality of pieces of element information into groups; and
generating summary information in which a summary of the element information included in the group is associated with the group.
